(19) United States
(12) Patent Application Publication
(10) Pub. No.: US 2024/0173115 A1
(43) Pub. Date: May 30, 2024

(54) INJECTABLE FILLER FROM AUTOLOGOUS DERMIS WITHOUT DONOR SCARRING

(71) Applicant: The General Hospital Corporation, Boston, MA (US)

(72) Inventors: Ying Wang, Winchester, MA (US); R. Rox Anderson, Boston, MA (US); William Farinelli, Danvers, MA (US); Joshua Tam, Andover, MA (US)

(21) Appl. No.: 18/551,349

(22) PCT Filed: Mar. 22, 2022
(86) PCT No.: PCT/US2022/021380

§ 371 Date: Sep. 19, 2023

Related U.S. Application Data
(60) Provisional application No. 63/164,473, filed on Mar. 22, 2021.
(60) Provisional application No. 63/215,505, filed on Jun. 27, 2021.

(51) Int. Cl. A61F 2/00; A61B 17/32; A61K 8/98; A61L 27/36; A61L 27/50; A61Q 19/08; A61B 17/00; A61B 17/322

(52) U.S. Cl. CPC A61F 2/0059; A61B 17/320068; A61K 8/985; A61L 27/362; A61L 27/3691; A61L 27/50; A61Q 19/08; A61B 2017/00969; A61B 2017/3225; A61K 2800/91; A61L 2400/06

(57) ABSTRACT

A system and method for providing an autologous bio-filler to a patient is provided. The method includes harvesting dermal columns from a harvesting site and mincing the dermal columns into microsegments, where the microsegments form the autologous bio-filler. The method also includes injecting the autologous bio-filler into a recipient site of the patient.

### CROSS-REFERENCE TO RELATED APPLICATIONS

This application is based on, claims priority to, and incorporates herein by reference in their entirety, U.S. Provisional Patent Application Ser. No. 63/164,473, filed on Mar. 22, 2021, and U.S. Provisional Patent Application Ser. No. 63/215,505, filed on Jun. 27, 2021.

### BACKGROUND

Dermal fillers are substances that are injected beneath the skin, and sometimes within the skin, for example, to restore lost volume, smooth lines and wrinkles, soften creases, enhance contours, and/or improve the appearance of scars. Generally, current dermal fillers are foreign materials that, when injected into a patient, bring the risk of unwanted tissue reactions from allergy, granulomas, infections, etc. In addition to these risks, fillers are available at a high cost and require repeated treatments. For example, hyaluronic acid, the most popular filler material in the United States, degrades in about three to six months, such that three treatments per year is typical to maintain a desired additional volume. The cost of a typical filler is usually about $300 per cubic centimeter and, with the addition of physician reimbursement costs, most patients pay about $2000 per year. On the other hand, while autologous dermal strips or sheets used in cosmetic or reconstructive volume rejuvenation have been described in the past, they have had a limited role due to donor-site morbidity, graft bulk, and the need for open-access incisions.

Therefore, there exists a clinical need for a safe and effective dermal filler that can maintain volume with fewer associated risks.

### SUMMARY

The systems and methods of the present disclosure overcome the above and other drawbacks by providing an autologous bio-filler that can be acquired from the dermis of a patient with minimal to no donor site scarring.

In accordance with one aspect, a method for providing an autologous bio-filler to a patient is provided. The method includes harvesting dermal columns from a harvesting site and mincing the dermal columns into microsegments, where the microsegments form the autologous bio-filler. The method also includes injecting the autologous bio-filler into a recipient site of the patient.

In accordance with another aspect, a system for providing an autologous bio-filler to a patient is provided. The system includes a coring needle and a needle syringe. The coring needle is configured to harvest a dermal column from a harvesting site of the patient. The coring needle includes internal cutters that mince the dermal column into microsegments as the dermal column travels through the coring needle, where the microsegments form the autologous bio-filler. The needle syringe is configured to inject the autologous bio-filler at a recipient site of the patient.

In accordance with yet another aspect, a system for providing an autologous bio-filler to a patient is provided. The system includes a harvesting device, a mincing device, and an injecting device. The harvesting device is configured to harvest at least one dermal column from a harvesting site of the patient. The mincing device is configured to mince the dermal column into microsegments, where the microsegments form the autologous bio-filler. The injecting device is configured to inject the autologous bio-filler into a recipient site of the patient.

The foregoing and other advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a schematic view of a system for harvesting and injecting a bio-filler according to some embodiments.

FIG. 2 is a view of the system of FIG. 1, according to some embodiments, with components configured to perform a parallel harvesting method.

FIG. 3 is a cross-sectional view of a harvesting device, according to some embodiments, for use with the system of FIG. 2, at a harvesting site.

FIG. 4 is perspective view of the harvesting device of FIG. 2 at a harvesting site.

FIG. 5 is a cross-sectional view of another harvesting device, according to some embodiments, for use with the system of FIG. 2, at a harvesting site.

FIG. 6 is a cross-sectional view of a combination harvesting and mincing device, according to some embodiments, for use with the system of FIG. 2.

FIG. 7 is a view of the system of FIG. 1, according to some embodiments, with components configured to perform a perpendicular harvesting method.

FIG. 8 is a cross-sectional view of a harvesting device, according to some embodiments, for use with the system of FIG. 7, at a harvesting site.

FIG. 9 is a cross-sectional view of a tip of a coring needle for use as a harvesting device in the system of FIG. 7, according to some embodiments.

FIG. 10 is a cross-sectional view of a harvesting device, according to some embodiments, for use with the system of FIG. 7, at a harvesting site, configured to perform an angled harvesting method.

FIG. 11 is a side view of a harvesting guide, according to some embodiments, for use with the system of FIG. 7 to perform an angled harvesting method.

FIG. 12 is a perspective view of another harvesting guide, according to some embodiments, for use with the system of FIG. 7 to perform an angled harvesting method.

FIG. 13 is a perspective view of yet another harvesting guide and a harvesting device, according to some embodiments, for use with the system of FIG. 7 to perform a perpendicular or angled harvesting method.

FIG. 14 is a top view of cored dermal columns obtained from harvesting with a system, of some embodiments, at a 90-degree angle, a 30-degree angle, a 15-degree angle, and a 10-degree angle.

FIG. 15 is a bar graph showing column length relative to harvesting angle of the cored dermal columns of FIG. 14.

FIG. 16 is cross-sectional views of curved coring needles, where FIG. 16A is a cross-sectional view of a U-shaped coring needle; FIG. 16B is a cross-sectional view of a J-shaped coring needle; and FIG. 16C is a cross-sectional view of a corkscrew-shaped coring needle.

FIG. 17 is a flow chart illustrating a method, according to some embodiments, for harvesting and injecting a bio-filler.

FIG. 18 is a graph showing filler volume over time of injected bio-filler, obtained and injected in accordance with some embodiments, and injected hyaluronic acid.

### DETAILED DESCRIPTION

The disclosure provides systems and methods for acquiring an injectable filler material for tissue augmentation by harvesting autologous micro-cored dermis. On the harvesting side, the systems and methods use coring needle harvesting methods, which avoid incisions and only cause needle-size wounds, which can heal without scarring. On the augmentation side, the systems and methods use autologous tissue as a filler material, which can be considered safe (since it replaces “like with like”), non-allergenic, noncarcinogenic, and nonteratogenic. Using autologous dermal tissue as filler material also provides a more robust mesenchymal cell construct than, for example, fat, so it can have better volume maintenance, be less likely to break down over time, and feel more like normal tissue compared to other current fillers.

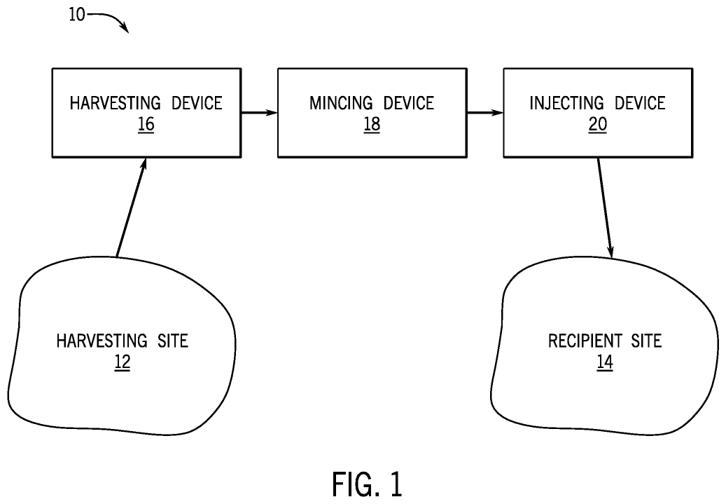

For example, FIG. 1 illustrates a schematic view of a system 10 for harvesting and injecting a bio-filler material, according to some embodiments. That is, the system 10 is configured to harvest dermis from a harvesting site 12 and use it as a bio-filler material to inject at a recipient site 14. As shown in FIG. 1, the system 10 can include a harvesting device 16, a mincing device 18, and an injecting device 20. The harvesting device 16 can be configured to harvest dermal tissue in the form of micro-cored dermal columns from the harvesting site 12, the mincing device 18 can be configured to mince the harvested dermis into a bio-filler material (e.g., an injectable micro-scale dermis), and the injecting device 20 can be configured to inject the bio-filler material into the recipient site 14. It should be noted that one or more of the harvesting device 16, the mincing device 18, and/or the injecting device 20 may be combined into a single device, rather than separate devices, such that the system 10 may include one, two, three, or more devices.

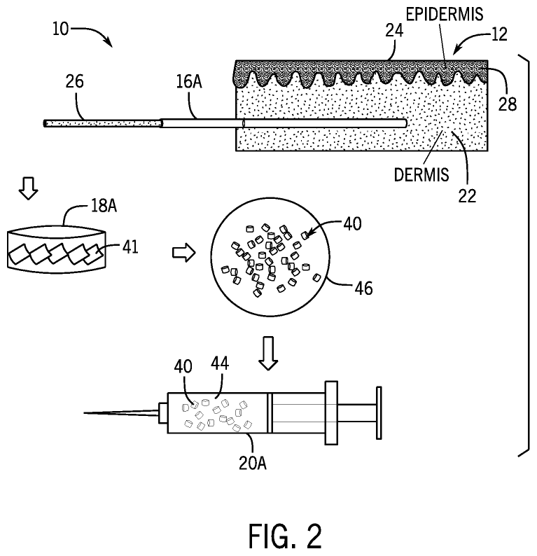

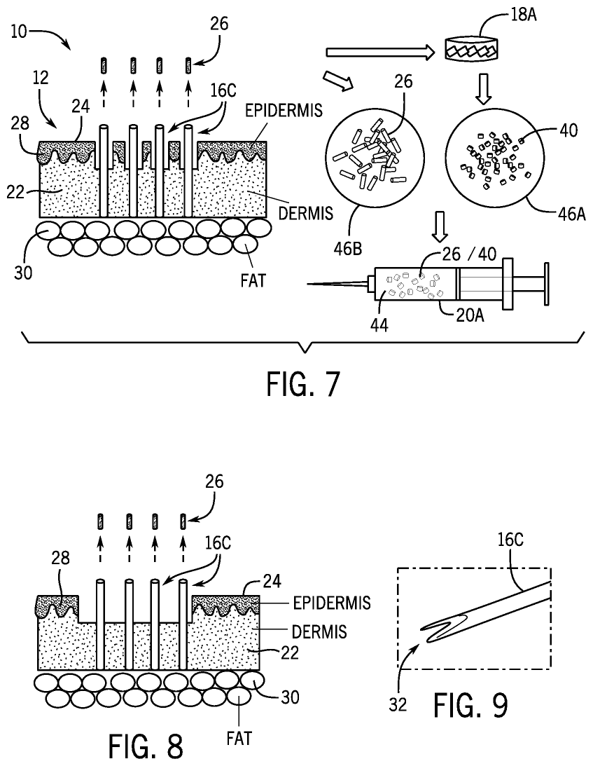

The system 10 can be configured to perform a parallel harvesting method, as shown and described with respect to FIGS. 2-6, a perpendicular harvesting method, as shown and described with respect to FIGS. 7-9, or a perpendicular harvesting method, as shown and described with respect to FIGS. 7 and 9-12. Generally, all of these harvesting methods allow a patient's own dermal tissue to be harvested from the harvesting site 12 (e.g., a donor site) in a manner that does not cause visible scarring, and allows the dermal tissue to be used as an injectable, autologous, living filler material at the recipient site 14. Also, it should be noted that any details or system components described herein with respect to one harvesting method can be used for another harvesting method even if not specifically described or shown. As one specific example, certain harvesting devices 16 described with respect to one harvesting method may be used for another harvesting method.

Referring now to FIG. 2, the system 10 according to some embodiments illustrated. As shown in FIG. 2, the system 10 can include a harvesting device 16 in the form of one or more coring needles 16A (e.g., micro-coring needles), a mincing device 18, and an injecting device 20 in the form of a needle syringe 20A. The system 10 can be used to perform a parallel harvesting method such that the coring needle 12A can be tunneled through the dermis 22 at a harvesting site 12, running parallel to the skin surface 24, to obtain a dermal core or column 26. As a result, the dermis 22 is accessed by making a small hole in the epidermis 28 and this needle-sized wound may be capable of healing without scarring.

For example, the diameter or width of the hole created by the coring needle 16A of the system 10 of FIG. 2 (or the harvesting devices 14 of any of the systems 10 described herein) can without limitation be less than about 1.0 millimeter (mm) and results in a dermal core 26 having a diameter or width that is less than about 1.0 mm. In further embodiments, the dermal core 26 has a diameter or width that is less than about 1.0 mm and a length of more than about 10.0 mm (e.g., about 10 mm, 20 mm, 30 mm, 40 mm or 50 mm, et al.). Accordingly, the relatively small dimensions of the dermal cores 26 can promote healing while minimizing the formation of scars.

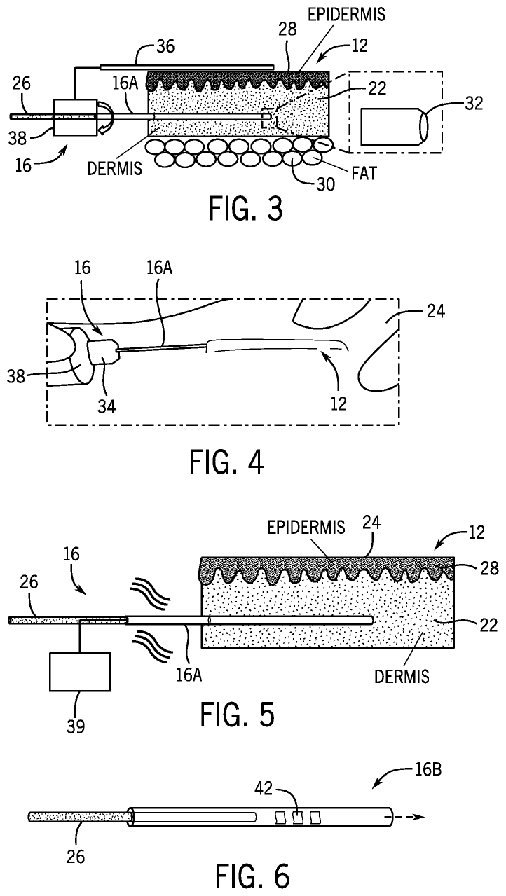

Furthermore, the coring needle 16A of the system 10 of FIG. 2 (or another coring needle of any of the systems 10 described herein) can be a micro-coring needle characterized as a hollow tube or needle with a sharp edge (e.g., where the inner diameter of at least one tube is less than about 1 mm, about 0.5 mm, about 0.3 mm, or about 0.2 mm). Some example needles can include: a 16 gauge needle having an inner diameter of 1.194 mm; an 18 gauge needle having an inner diameter of 0.838 mm; a 20 gauge needle having an inner diameter of 0.564 mm; a 23 gauge needle having an inner diameter of about 0.337 mm and an outer diameter of about 0.51 mm, thereby resulting in a dermal core 26 having a dimension (e.g., a width or diameter) of about 0.3 mm; a 25 gauge needle having an inner diameter of about 0.26 mm or a thin-walled 25 gauge needle having an inner diameter of about 0.31 mm and an outer diameter of about 0.51 mm, thereby resulting in a dermal core 26 having a dimension (e.g., a width or diameter) of about 0.2 mm; a 30 gauge needle having an inner diameter of about 0.159 mm; a 32 gauge needle having an inner diameter of about 0.108 mm; or a 34 gauge needle having an inner diameter of about 0.0826 mm. In some embodiments, the coring needle 16A of the system of FIG. 2 can be an edged coring needle, for example, including a substantially blunt end with a circular edge 32, as shown in FIG. 3. In some embodiments, the coring needle 16A can comprise a two-point edged coring needle (e.g., having an end 32 with two points), or can comprise a multi-point edged coring needle (e.g., having an end 32 with two or more than two points). In further embodiments, the coring needle 16A can be a biopsy punch. Additionally, in some embodiments, the coring needle 16A can be lubricated with sterile lubricants (e.g., hydrogel, Vaseline, or other lubricants) and can be driven slowly to avoid thermal damage.

When a desired length of cored dermis 26 has been reached, the coring needle 16A can be directed into the subcutaneous fat 30 to allow complete extraction of the dermal core 26 (e.g., due to the fact that fat cleaves easily). The coring needle 16A can be adapted for a single extraction, e.g., to obtain one dermal core 26, or can be adapted to obtain more than one dermal core 26. More specifically, a single dermal core 26 may be extracted into the coring needle 16A, or multiple passes through the dermis 22 can be completed so that multiple dermal cores 26 can be obtained within a single coring needle 16A. In this manner, the coring needle 16A can act as a reservoir for one or more harvested dermal cores 26.

Additionally, in some embodiments, the coring needle 16A can rotate or vibrate in order to tunnel through the dermis 22. For example, an electric motor 34 can be coupled to an end of the coring needle 16A (e.g., directly or via a holding device or needle support) in order to rotate the coring needle 16A as it is driven through the dermis 22 parallel to the skin surface 24, as shown in FIGS. 3 and 4. As another example, a vibrating device 39 can be coupled to the coring needle 16A, as shown in FIG. 5, in order to vibrate the coring needle 16A at a frequency sufficient to move the coring needle 16A through the dermis 22 but not impart heating to the skin. For example, vibration of the coring needle 16A may be longitudinal, and may be driven by the vibrating device 39 in the form of a motor, electromagnetic coil, piezoelectric driver, or other mechanical activators, and may be at a resonant frequency. Additionally, in some embodiments, to prevent overheating, the coring needle 16A and/or the patient's skin may be actively cooled (e.g., by the harvesting device 16 or a separate device, such as a cold air cooling device).

Furthermore, in some embodiments, the harvesting device 16 can include a guide 36, as shown in FIG. 3, configured to help guide the coring needle 16A through the dermis 22. For example, the guide 36 can be coupled to and extending from a needle support 36 of the coring needle 16A, and be configured to move parallel to the coring needle 16A across the skin surface 24 (e.g., touching the skin surface 24 or raised above the skin surface 24). As such, the guide 36 can help keep and guide the coring needle 16A parallel to the skin surface 24 as it cuts through the dermis 22.

Referring back to FIG. 2, the long dermal cores 26 that are acquired by the harvesting device 16 can be minced into microsegments of dermal fragments 40 using the mincing device 18. In some embodiments, the mincing device 18 imparts mechanical disruption to the dermal cores 26, such as by sharp mincing, grinding, tissue homogenizing, or ultrasound-induced cavitation. Accordingly, the mincing device 18 can comprise, for example, scalpel blades or other movable blades, a tissue homogenizer, an electrical trimmer, electrical scissors, and/or a cavitation device. In one example, as shown in FIG. 2, the mincing device 18 can be a mechanical mincing device 18A having movable blades 41. In further embodiments, the mincing device 18 can be a mortar and pestle arrangement, a pill grinder arrangement, a pulverizer, or another device configured to grind or crush the dermal cores 26. Additionally, in some embodiments, the dermal cores 26 may be frozen prior to mechanical disruption to facilitate fragmentation.

In some embodiments, the mincing device 18 can be coupled directly to the harvesting device 16 such that mechanical disruption is imparted at the time of harvesting (e.g., the harvested dermal cores 26 are moved directly into the mincing device 18 from the coring needle 16A). In other embodiments, the mincing device 18 is combined into the harvesting device 16. For example, as shown in FIG. 6, in some embodiments, a coring needle 16B can include rotary cutters or grinding elements 42 to mince the cored dermal tissue 26 as it is directed into and/or travels through the coring needle 16B. In yet other embodiments, the mincing device 18 is separate from the harvesting device 16 such that the harvested dermal cores 26 are extracted from the coring needle 20A and placed into the mincing device 18 to be minced. Accordingly, mincing can be accomplished at the time of harvesting or after harvesting.

In some embodiments, the above-described mechanical disruption of the dermal cores 26 may kill part of dermal cells within the dermal cores 26. In other embodiments, most of cell viability is retained such that mechanical disruption only affects the superficial dermal cells in the bio-filler. Additionally, though mechanical disruption is described and illustrated herein, it is within the scope of some embodiments to incorporate chemical or enzymatic processes as an addition or alternative to mechanical disruption.

Referring back to FIG. 2, from the mincing device 18, the microsegments 40 can be collected by the injecting device 20, such as a needle syringe 20A, e.g., with an appropriate volume of sterile fluid 44. For example, in some embodiments, the minced microsegments 40 can be placed in a sterile dish 46 with a volume of sterile fluid 44. The needle syringe 20A can collect the microsegments 40 and fluid 44 from the dish 46. Alternatively, in some embodiments, the sterile fluid 44 can be mixed with the microsegments 40 within the mincing device 18 and either extracted onto the dish 46 for collection by the needle syringe 20A or collected directly from the mincing device 18 by the needle syringe 20A. The injecting device 20 can then be used to inject the micro dermal segments 40, in the sterile fluid 44, directly into the recipient site 14 (e.g., subcutaneously, into the skin, or into the tissue) as a bio-filler material.

Referring now to FIG. 7, the system 10 according to some embodiments is illustrated. As shown in FIG. 7, the system 10 can include a harvesting device 16 in the form of one or more coring needles 16C, a mincing device 18, and an injecting device 20 in the form of a needle syringe 20A. The system 10 can be used to perform a perpendicular harvesting method such that the coring needle(s) 16C can be inserted vertically into the dermis 22, i.e., running perpendicular to the skin surface 24, to obtain one or more dermal cores 26. As a result, the dermis 22 is accessed by making one or more small holes and these needle-sized wounds may be capable of healing without scarring.

Additionally, in some embodiments, prior to harvesting, the epidermis 28 of the harvesting site 12 can be removed so that only dermis 22 (and subcutaneous fat 30) can be harvested, as further discussed below. More specifically, in some embodiments, the harvesting site 12 can be prepared in that the epidermis 28 can be removed at each needle site within the harvesting site 12, as shown in FIG. 7, or a full area of the epidermis 28 within the harvesting site 12 can be removed, as shown in FIG. 8. The epidermis 28 can be removed by, for example, a dermal blade, debridement, suction blistering, or other methods.

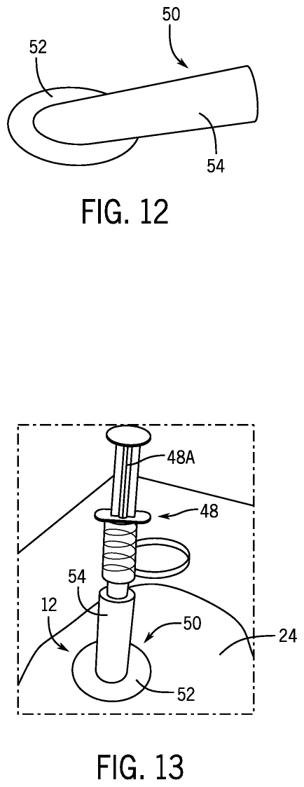

In some embodiments, once the harvesting site 12 is prepared, the coring needle 16C can be inserted a depth through the dermis 22 until reaching the subcutaneous fat 30. Accordingly, the dermis 22 as well as a small amount of subcutaneous fat 30 can be harvested. In some embodiments, the coring needle 16C can comprise a two-point edged coring needle 16C, as shown in FIG. 9. In some embodiments, the coring needle 16C can include more than two points. In further embodiments, the coring needle 16C can be a biopsy punch. The coring needle 16C can be used, with suction, to extract the dermal cores 26 (e.g., to pull the dermal cores 26 vertically up into the coring needle 16C via suction). In some embodiments, a suction device can be connected to the coring needle 16C to apply suction necessary to extract the dermal cores 26. For example, the suction device may be a syringe 48 (as shown in FIG. 12) that is manually or automatically actuated to apply the necessary suction to extract the dermal cores 26. Additionally, in some embodiments, the coring needle 16C can be a single coring needle 16C, can be a plurality of separate coring needles 16C, or can be an array of coring needles 16C. For example, in some embodiments, the system 10 can be provided as a kit (e.g., a single-use or multi-use kit) having a single coring needle 16C, a plurality of separate coring needles 16C, or an array of coring needles 16C.

Referring back to FIG. 7, the long dermal cores 26 that are acquired by the coring needle 16C can be minced into microsegments of dermal fragments 40 using a mincing device 18, such as any of the devices illustrated and described above with respect to the system 10 of FIG. 2. Additionally, in some embodiments, the system 10 may not include a mincing device 18. Rather, the whole dermal cores 26 may be used as a dermal filler without mechanical disruption.

Referring still to FIG. 7, from the mincing device 18 or directly from the coring needles 16C, the microsegments 40 can be collected by the injecting device 20, such as a needle syringe 20A, e.g., with an appropriate volume of sterile fluid 44 (e.g. saline, Ringer's solution, or hyaluronic acid). For example, in some embodiments, the minced microsegments 40 can be placed in a dish 46A with a volume of sterile fluid 44, or the whole cored dermal segments 26 can be placed in a dish 46B with a volume of sterile fluid 44, as shown in FIG. 7. The needle syringe 20A can collect the microsegments 40 or full core segments 26 and fluid 44 from the dish 46. Alternatively, in some embodiments, the sterile fluid 44 can be mixed with the microsegments 40 within the mincing device 18 and either extracted onto the dish 46 for collection by the needle syringe 20A or collected directly from the mincing device 18 by the needle syringe 20A. The injecting device 20 can then be used to inject the micro dermal segments 40 (or whole core segments 26), in the sterile fluid 44, directly into the recipient site 14 (e.g., subcutaneously, into the skin, or into the tissue) as a bio-filler material.

As noted above, in some embodiments, the epidermis 28 at the harvesting site 12 may be removed prior to harvesting. This can prevent encapsulated epidermal components causing an inflammatory response at the recipient site 14. More specifically, a study was conducted comparing minced dermal columns (i.e., including only dermis and, potentially, some subcutaneous fat) and minced skin tissue columns (i.e., including epidermal, dermis and, potentially, some subcutaneous fat). Both the dermal columns and the skin tissue columns were mixed with a sterile saline and 0.5 milliliters (mL) of the mixtures were injected subcutaneously into a swine's belly area. The recipient sites were analyzed at day zero and week six, and histological evaluations were performed at week six. At week six, no inflammatory reaction was observed from the minced dermal column injection. On the other hand, a high inflammatory reaction was observed from the skin tissue column injection, which appeared to be induced by encapsulated epidermal cells and stratum corneum.

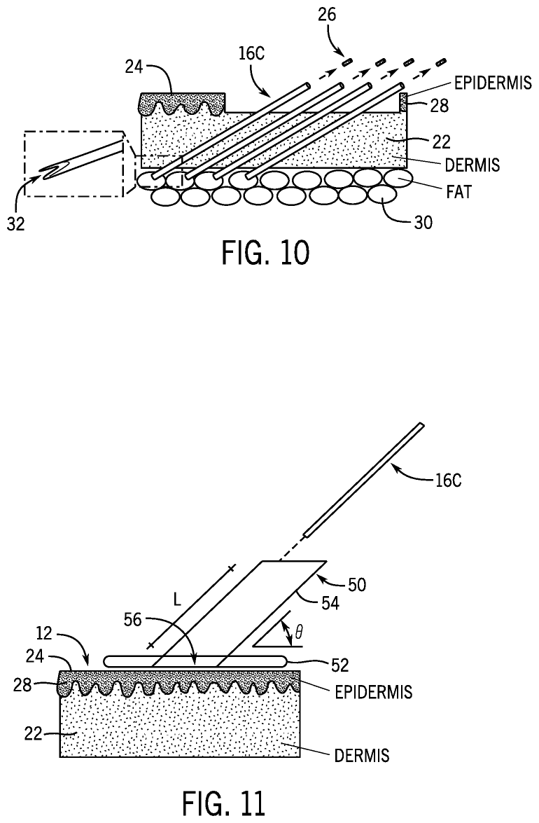

Referring now to FIG. 10, in some embodiments, the system 10 of FIG. 7 can be used to perform an angle harvesting method such that the coring needle(s) 16C can be inserted into the dermis 22 at an angle relative to the skin surface 24 to obtain one or more dermal cores 26. As a result, the dermis 22 is accessed by making one or more small holes and these needle-sized wounds may be capable of healing without scarring.

In such embodiments, as shown in FIG. 10, the system 10 can include a two-point edged coring needle 16C. Additionally, in some embodiments, the system 10 can include a harvesting guide 50, as shown in FIGS. 11, 12, and 13, configured to guide the coring needle 16C into and through the dermis 22. For example, as shown in FIG. 10, the harvesting guide 50 can include a base 52 configured to rest upon the patient's skin surface 24 and an angled portion 54 configured to extend from the base 52 at a harvesting angle θ. The base 50 can include a hole 56 therethrough and the angled portion 54 can be hollow and align with the hole 56 so that the coring needle(s) 16C can be routed through the harvesting guide 50 (i.e., through the hollow angled portion 54 and the hole 56 of the base 52) so that the coring needle 16C is directed into the dermis 22 at the harvesting angle θ.

FIG. 12 illustrates the harvesting guide 50 having a first harvesting angle θ, and FIG. 13 illustrates the harvesting guide 50 having a second harvesting angle θ larger than the first harvesting angle θ (e.g., as measured relative to the skin surface 24). In some embodiments, the harvesting angle θ can be between about 0 degrees and about 90 degrees or between about 0 degrees and up to, but not including about 90 degrees. In some embodiments, the harvesting angle θ can be between about 5 degrees and about 85 degrees, between about 10 degrees and about 80 degrees, between about 15 degrees and about 75 degrees. Accordingly, the harvesting guide 50 can be used for angled or perpendicular harvesting methods.

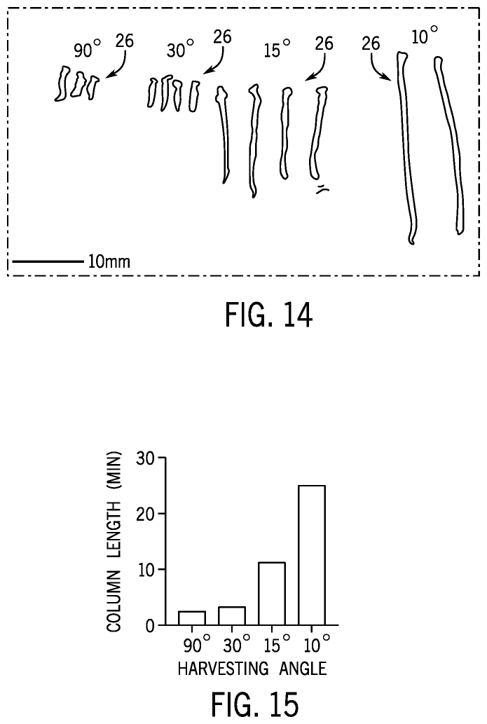

In some embodiments, the harvesting angle θ can affect a total length of dermal core 26 that can be extracted from the dermis 22. For example, FIG. 14 illustrates dermal cores 26 extracted at 90-degree, 30-degree, 15-degree, and 10-degree harvesting angles θ (i.e., measured relative to the skin surface 24) and FIG. 15 is a graph that illustrates core length of the dermal cores 26 of FIG. 13 (in millimeters) relative to harvesting angle θ (in degrees). As shown in FIGS. 14 and 15, a smaller harvesting angle θ may permit extraction of a longer dermal core 26. More specifically, the 10-degree harvesting angle θ resulted in the longest extracted dermal cores 26 while the 90-degree harvesting angle θ (i.e., a perpendicular harvesting method) resulted in the shortest extracted dermal cores 26. That, is, using the 10-degree harvesting angle θ could harvest more than five times longer dermal cores 26 than using the 90-degree harvesting angle θ.

Additionally, referring back to FIG. 11, in some embodiments, the angled portion 54 of the harvesting guide 50 can also include a specific length L sized to help guide the coring needle(s) 16C to a desired depth within the dermis 22 and/or the subcutaneous fat 30. The length L can also be sized to permit a desired suction device 48 to be coupled to the coring needle 16C in order to apply suction to extract the dermal core 26. For example, FIG. 13 illustrates the coring needle 16C (within the angled portion 54 of the harvesting guide 50) coupled to a spring-loaded syringe 48A that can apply suction to pull a dermal core 26 into the coring needle 16C.

Once the dermal cores 26 are extracted using the angled harvesting method, the dermal cores 26 can be minced with a mincing device 18 (or left whole) and applied to an injecting device 20 in order to inject the micro dermal segments 40 (or whole dermal segments 16), in a sterile fluid (e.g. saline, Ringer's solution or hyaluronic acid) 44, directly into the recipient site 14 (e.g., subcutaneously, into the skin, or into the tissue) as a bio-filler material, as described above with respect to the systems of FIG. 2 or 7.

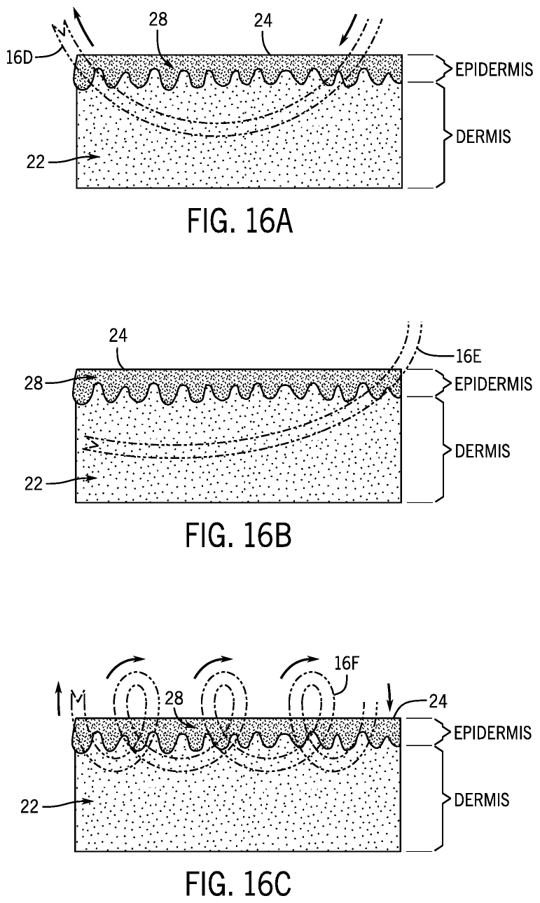

The parallel, perpendicular, and angled harvesting methods are shown and described above using coring needles 16A, 16B, 16C that are straight. However, in some embodiments, any of these methods may utilize a curved coring needle as part of the harvesting device 16. For example, FIG. 16A illustrates a curved coring needle 16D that is substantially U-shaped such that it can poke back out of the skin surface 24. FIG. 16B illustrates a curved coring needle 16E that is substantially J-shaped such that it can extend through the skin surface 24 and travel substantially parallel through the dermis 22. FIG. 16C illustrates a curved coring needle 16F that is corkscrew shaped such that it can harvest multiple dermal columns 26 with one insertion.

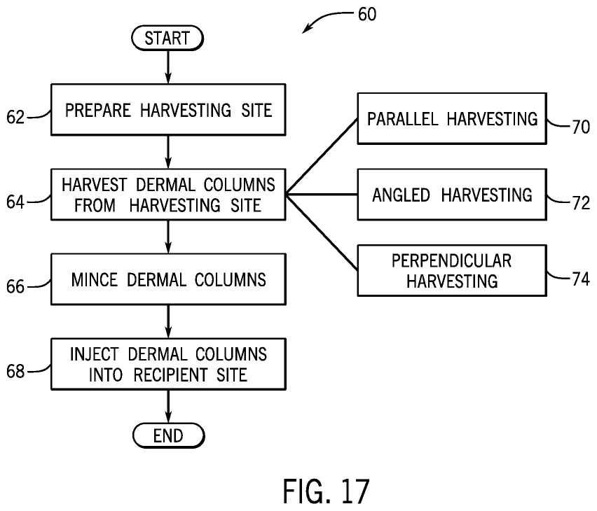

In light of the above, FIG. 17 illustrates a method 60 for acquiring and/or injecting an autologous bio-filler, according to some embodiments. Generally, the method can include preparing the harvesting site 12 (step 62), harvesting dermal columns from the harvesting site 12 (step 64), mincing the harvested dermal columns (step 66), and injecting the dermal columns into a recipient site 14 (step 68). It should be noted that, in some embodiments, the method 60 may not include all steps, may include steps combined together, may include steps in a different order than that shown in FIG. 17, and/or may include additional steps. In some embodiments, the method 60 can be performed on a patient as a “bedside” procedure, for example, in an outpatient or clinic setting or in a hospital setting. With further reference to the method of FIG. 17, step 62 includes preparing the harvesting site 12. The harvesting site 12 can be an area of the patient's skin. For example, the bio-filler in some embodiments is autologous and, thus, comes from the patient. Alternatively, in some embodiments, the harvesting site 12 may be an area of a donor's skin (e.g., a person or animal separate from the patient). In some embodiments, preparing the harvesting site 12 at step 62 includes cleaning the patient's skin at the harvesting site 12. In further embodiments, preparing the harvesting site 12 includes removing the epidermis 28 at the harvesting site 12. For example, as described above with respect to the perpendicular harvesting method, the epidermis 26 at the harvesting site 12 can be removed by a dermal blade, debridement, suction blistering, or other methods. Alternatively, in some embodiments, no preparation is necessary and step 62 may be skipped.

Step 64 includes harvesting dermal columns 26 from the harvesting site 12. Step 64 can be executed using a harvesting device 16 via a parallel harvesting method 70, a perpendicular harvesting method 72, and/or an angled harvesting method 74. For example, the harvesting device 16 can be a coring needle 16A/16B/16C, or a plurality of coring needles 16A/16B/16C, as described above with respect to the systems 10 of FIGS. 2-12. More specifically, one or more coring needles 16A/16B/16C can be inserted into the dermis 22, generally parallel to the skin surface 24 (according to a parallel harvesting method 70), generally perpendicular to (90 degrees relative to) this skin surface 24 (according to a perpendicular harvesting method 72), or at an angle between about zero degrees and about 90 degrees relative to the skin surface 24 (according to an angled harvesting method 74), to extract a length of dermal core 26, including dermis 22 or dermis 22 and some subcutaneous fat 30.

Step 66 includes mincing the harvested dermal columns 26. Step 66 can be executed using a mincing device 18, such as one of the mincing device 18 examples described above with respect to the systems 10 of FIGS. 2-12. Generally, mincing can include breaking down the long dermal cores 26 into microsegments through, for example, mechanical disruption. The microsegments may be, for example, less than about 300 micrometer (μm) length segments, sized to fit through a needle bore of a 21-gauge needle or less. In another example, the microsegments may be about 200 μm to about 600 μm, or may be less than about 600 μm. In a further example, the microsegments may be sized to fit through a needle bore of a needle between 18 gauge and 26 gauge. Alternatively, the dermal cores 26 can be broken into microsegments via chemical or enzymatic processes. In some embodiments, the dermal cores 26 are first removed from the harvesting device 16 after step 64 and applied to the mincing device 18 at step 66 to be minced.

In other embodiments, the harvesting device 16 and the mincing device 18 are coupled together so that the dermal cores 26 harvested at step 64 are directly applied from the harvesting device 16 to the mincing device 18 for mincing at step 66. In further embodiments, step 66 can be combined with step 64, e.g., using a combination harvesting and mincing device. For example, such embodiments can include a coring needle 16B with rotary cutters or grinding elements 42, as shown in FIG. 6, to mince the cored dermal tissue 26 as it is directed into the coring needle 16B. In any of the above embodiments, step 66 may be performed immediately after step 64 or there may be a time period between steps 64 and 66.

Generally, the acquired microsegments 40 can form an autologous bio-filler for injection. In some embodiments, step 66 can include further preparing the microsegments 40 as the autologous bio-filler. For example, preparing the microsegments 40 can include mixing them with a volume of sterile fluid 44. In some embodiments, the sterile fluid 44 (e.g. saline, Ringer's solution or hyaluronic acid) can be added to the mincing device 18 before or after applying the dermal columns 26 to the mincing device 18. Additionally, in some embodiments, the minced dermal columns 40 and a sterile fluid 44 can be added to a dish 46, as shown in FIGS. 2 and 7, either separately or after being mixed together. Furthermore, in some embodiments, further preparing the microsegments 40 can also include deliberately killing the cells in the harvested tissue, inducing desired biologic changes, e.g., by treating with anti-inflammatory agents or with genetic engineering tools, and/or altering the physical property of the bio-filler, e.g., by heating or chemical cross-linking.

In some embodiments, the method may not include step 66, such that the bio-filler includes larger segments of extracted dermal columns 26, e.g., directly from the harvesting device 16 without mechanical disruption, rather than microsegments 50. In such embodiments, the larger segments of dermal cores 26 may be prepared at step 64 or step 68. That is, the larger segments of dermal cores 26 may be prepared as a bio-filler by being mixed with a sterile fluid 44.

Step 68 includes injecting the dermal columns (i.e., the autologous bio-filler) into a recipient site 14. Step 68 can be executed by an injecting device 20, such as a needle syringe 20A, as described above with respect to the systems 10 of FIGS. 2-12. More specifically, a user (such as a medical provider, clinician, nurse, non-physician provider, etc.) can acquire a volume of bio-filler from the harvesting device 16, the mincing device 18, or a dish 46 into the needle syringe 20A, and can inject the bio-filler into the recipient site 14.

The recipient site 14 may be a subcutaneous site in some applications (e.g., such that the bio-filler is used as a dermal filler in the face, hands, or other body part of the patient), or may be skin or other internal tissue sites in some applications, such as dental tissue, vocal cord tissue, tendons, or other soft tissues. Accordingly, the method 60 of some embodiments may be used for cosmetic purposes and/or therapeutic purposes. For example, the bio-filler may be used as a dermal filler for volume restoration, wrinkle and crease removal or reduction, skin smoothing, contouring, reducing the appearance of acne scars, etc. In another example, the bio-filler may be injected into the skin and/or under the skin, for example, to fill a depressed scar, atrophic skin lesions, etc. In yet another example, the bio-filler may be injected into other tissues to help with, e.g., vocal fold paralysis, laryngoplasty, dental soft-tissue augmentation, soft-tissue defects, depressed scars, rhinoplasty, lip augmentation, cleft lip repair, stress urinary incontinence, tendon repair, among other applications. Additionally, long dermal columns 26 may be used as a bio-filler in some applications (such as dental soft-tissue augmentation, soft-tissue defects, depressed scars, stress urinary incontinence, tendon repair, or other applications), whereas minced dermal segments 40 may be used in other applications (such as dermal fillers, vocal fold fillers, laryngoplasty, dental soft-tissue augmentation, soft-tissue defects, depressed scars, rhinoplasty, or other applications).

After step 68, the method 60 may be complete, or the method 60 may be repeated at the same or a different recipient site 14. In some applications, however, one injection may provide a permanent filler solution, or a longer-term filler solution compared to present commercial fillers. For example, in one study, harvested swine dermal columns, acquired using the systems 10 and parallel harvesting methods 60 described herein, were analyzed and compared to hyaluronic acid, a current commercial filler material, and shown to maintain filler volume for an extended period of time compared to hyaluronic acid. In the study, the acquired swine dermal columns were minced and mixed with a sterile saline, in accordance with methods described herein, and 0.5 mL of the bio-filler (minced dermal columns, MDCs) and of hyaluronic acid (HA) were separately injected subcutaneously into swine ears ex vivo and in vivo. Volume measurement as well as histology (e.g., H&E stain, trichome stain, herovici stain) were analyzed.

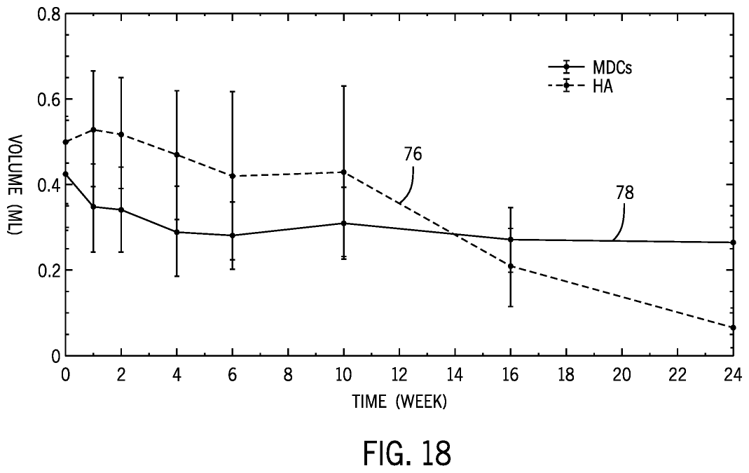

First, inspection of the harvested dermal columns illustrated collagen bundles. Once minced and injected, the volume of bio-filler (MDCs) and HA was measured over 24 weeks after injection. As shown in FIG. 18, HA volume, shown at line 76, dropped dramatically after week ten, while MDC volume, shown at line 78, was generally maintained over the 24-week monitoring period. Additionally, histology analysis at week eight showed vacuoles of HA, whereas the injected MDCs survived as a viable unit, exhibiting viable fibroblasts, collagen persistence, organization, and neovascularization. Histology analysis at week 24 showed that HA could no longer be detected, whereas the injected MDCs still maintained volume and still survived as a viable unit, exhibiting viable fibroblasts, collagen persistence, organization, and neovascularization.

Additionally, as noted above, the present systems 10 and methods 60 can harvest dermal tissue as an autologous bio-filler while minimally impacting the harvesting site 12. For example, in the above study, the harvesting site 12 was also analyzed. That is, gross images of the harvesting site 12 were analyzed at day zero, and weeks two, four, six, ten, and sixteen. The analysis showed re-epithelization after one week, and hyperpigmentation was observed throughout. Furthermore, tissue staining showed newly formed collagen at the harvesting site 12. Accordingly, the systems 10 and methods 60 described herein can result in minimum long-term impact to the harvesting site 12, including quick healing and no or minimal scarring.

In light of the above, some embodiments provide systems and methods for harvesting and mincing dermal tissue for acquiring and injecting an autologous bio-filler. These systems and methods have practical, economic, and safety advantages over present filler materials. For example, an autologous dermal filler may be considered safer than present fillers because it is not a foreign material being injected into the patient. That is, using autologous tissue as a filler material can be considered safer than current filler materials since it replaces “like with like.” Furthermore, the autologous bio-filler of some embodiments can create a more natural tissue outcome for aesthetic and reconstructive soft tissue augmentation procedures. Additionally, the autologous dermal filler, as acquired using the systems and methods described herein, is less prone to breakdown, does not degrade over time and, as a result, may be a permanent filler solution. For those reasons, it may be an economically attractive alternative to present filler materials, which are expensive and only provide temporary results. The long-term volume maintenance makes it a viable alternative for both cosmetic and therapeutic purposes.

The present invention has been described in terms of one or more preferred embodiments, and it should be appreciated that many equivalents, alternatives, variations, and modifications, aside from those expressly stated, are possible and within the scope of the invention. Furthermore, the term “about” as used herein means a range of plus or minus 20% with respect to the specified value, more preferably plus or minus 10%, even more preferably plus or minus 5%, most preferably plus or minus 2%. In the alternative, as known in the art, the term “about” indicates a deviation, from the specified value, that is equal to half of a minimum increment of a measure available during the process of measurement of such value with a given measurement tool.

1. A method for providing an autologous bio-filler to a patient, the method comprising:
harvesting dermal columns from a harvesting site;
mincing the dermal columns into microsegments, the microsegments forming the autologous bio-filler; and
injecting the autologous bio-filler into a recipient site of the patient.

2. The method of claim 1 and further comprising mixing the microsegments with a sterile fluid to form the autologous bio-filler.

3. The method of claim 1, wherein harvesting the dermal columns includes guiding a coring needle through a dermis of the patient substantially parallel to a skin surface of the patient.

4. The method of claim 3, wherein harvesting the dermal columns further includes rotating the coring needle as it is guided through the dermis.

5. The method of claim 3, wherein harvesting the dermal columns further includes vibrating the coring needle as it is guided through the dermis.

6. The method of claim 1, wherein harvesting the dermal columns includes guiding a coring needle through a dermis of the patient substantially perpendicular to a skin surface of the patient.

7. (canceled)

8. The method of claim 6, wherein harvesting the dermal columns further includes applying suction to the coring needle to pull one or more of the dermal columns into the coring needle.

9. The method of claim 1 and further comprising removing an epidermis of the patient at the harvesting site prior to harvesting the dermal columns.

10. The method of claim 1, wherein harvesting the dermal columns includes harvesting the dermal columns using a coring needle having internal cutters, and mincing the dermal columns includes pulling the dermal columns through the coring needle.

11. A system for providing an autologous bio-filler to a patient, the system comprising:
a coring needle configured to harvest a dermal column from a harvesting site of the patient, the coring needle including internal cutters that mince the dermal column into microsegments as the dermal column travels through the coring needle, the microsegments forming the autologous bio-filler; and
a needle syringe configured to inject the autologous bio-filler at a recipient site of the patient.

12. (canceled)

13. (canceled)

14. The system of claim 11 and further comprising a harvesting guide configured to guide the coring needle into a dermis of the patient to harvest the dermal column.

15. The system of claim 11 and further comprising a motor coupled to the coring needle and configured to rotate the coring needle.

16. The system of claim 11 and further comprising a vibrating device coupled to the coring needle and configured to vibrate the coring needle.

17. (canceled)

18. A system for providing an autologous bio-filler to a patient, the system comprising:
a harvesting device configured to harvest a dermal column from a harvesting site of the patient;
a mincing device configured to mince the dermal column into microsegments, the microsegments forming the autologous bio-filler; and
an injecting device configured to inject the autologous bio-filler into a recipient site of the patient.

19. The system of claim 18, wherein the harvesting device is a coring needle.

20. The system of claim 18, wherein the injecting device is a needle syringe.

21. The system of claim 19, wherein the coring needle is one of a two-point edged or a multi-point edged coring needle.

22. The system of claim 18 and further comprising a sterile fluid mixed with the microsegments to form the autologous bio-filler.

23. The system of claim 19 and further comprising a guide configured to guide the coring needle into a dermis of the patient to harvest the dermal column.

24. (canceled)

25. The system of claim 23, wherein the guide includes a base configured to rest upon a skin surface of the patient and an angled portion extending from the base, the guide being sized to permit the coring needle to be guided through the guide at a harvesting angle.

26. The system of claim 19 and further comprising a motor coupled to the coring needle and configured to rotate the coring needle.

27. The system of claim 19 and further comprising a vibrating device coupled to the coring needle and configured to vibrate the coring needle.
